(19) United States
(12) Patent Application Publication
(10) Pub. No.: US 2026/0196351 A1
(43) Pub. Date: Jul. 9, 2026

(54) SYSTEMS AND METHODS FOR BEHAVIORAL HEALTH ANALYSIS USING ARTIFICIAL INTELLIGENCE

(71) Applicants: Wyatt Deane, Anaheim, CA (US); Jorge Eduardo, Las Vegas, NV (US); Lloyd Gilbert, Goodyear, AZ (US)

(72) Inventors: Wyatt Deane, Anaheim, CA (US); Jorge Eduardo, Las Vegas, NV (US); Lloyd Gilbert, Goodyear, AZ (US)

(21) Appl. No.: 19/438,947

(22) Filed: Jan. 2, 2026

Related U.S. Application Data
(60) Provisional application No. 63/742,087, filed on Jan. 6, 2025.

(51) Int. Cl. G16H 50/20

(52) U.S. Cl. CPC G16H 50/20

(57) ABSTRACT

A computer-implemented approach generates applied behavior analysis reports by aggregating structured and unstructured clinical and behavioral data, normalizing formats, and removing personally identifiable information before processing. One or more processors execute natural language processing to tokenize, lemmatize, tag parts of speech, and recognize entities, then compute contextual embeddings with a transformer-based model to capture relationships among behaviors, goals, interventions, and outcomes. The processors optionally construct and query a knowledge graph, evaluate outputs for bias, and adjust recommendation scoring. A template selection routine binds fields to variables and auto populates individualized goals, intervention steps, measurement criteria, and guidance. A user interface accepts corrections that the system stores for iterative refinement, and reports are persisted for retrieval and periodic progress updates. Related embodiments cover a computing system and a non-transitory computer readable medium storing instructions to perform the foregoing operations.

### CROSS-REFERENCE TO RELATED APPLICATIONS

The present application claims priority to U.S. Provisional Application No. 63/742,087 filed Jan. 6, 2025, titled “SYSTEMS AND METHODS FOR BEHAVIORAL HEALTH ANALYSIS USING ARTIFICIAL INTELLIGENCE,” which is hereby incorporated by reference in its entirety. This application further discloses embodiments involving immersive augmented-and virtual-reality behavioral capture systems, passive environmental sensing systems, and hybrid classical-quantum computational architectures. These embodiments form part of the originally contemplated invention and are supported throughout the present specification.

### TECHNICAL FIELD

The embodiments generally relate to systems and methods for computer-implemented applied behavior analysis, and more particularly to multimodal behavioral-health informatics systems that integrate machine learning models, immersive augmented-and virtual-reality interfaces, passive environmental sensing, and hybrid classical-quantum computational architectures. The disclosed embodiments involve the automated generation of individualized intervention plans, behavior analysis reports, and evidence-linked recommendations produced from structured clinical data, unstructured narrative inputs, sensor-derived behavioral observations, and optimization routines executed on classical and, in certain embodiments, quantum computing resources.

### BACKGROUND

Conventional behavioral health software platforms typically retrieve structured clinical information from electronic health records and may perform limited natural language processing on unstructured notes. These systems often rely on manual data entry, rule-based assessments, or static templates that do not adapt to variations in behavioral presentation across settings, sessions, or developmental profiles. Existing approaches generally lack mechanisms for integrating multimodal observational data, such as real-time behavioral signals captured through augmented- or virtual-reality interfaces or passive environmental sensors, with higher-level clinical reasoning models.

Prior tools for applied behavior analysis further lack automated methods for generating individualized intervention plans that incorporate evidence from heterogeneous inputs such as structured assessments, unstructured narrative notes, sensor-derived behavioral measures, or historical case outcomes. Emerging immersive therapeutic systems may provide virtual environments for skills training, but they do not tightly couple sensor-derived behavioral observations with machine-learned embeddings, knowledge graph representations, or iterative model-based report generation workflows. These systems also do not provide mechanisms for fusing immersive interaction data with longitudinal case records to support automated goal selection, intervention sequencing, or measurement system alignment.

Additionally, existing recommendation engines and decision-support platforms rely primarily on classical computing architectures, which may be insufficient to efficiently evaluate large combinatorial spaces involving competing clinical priorities, multi-goal optimization, or constraints imposed by payor, regulatory, or program requirements. Current systems do not provide hybrid classical-quantum computational workflows capable of performing constrained optimization or selection of tasks over high-dimensional behavioral and contextual features.

Accordingly, there remains a need for improved systems and methods that (i) ingest structured, unstructured, and sensor-derived behavioral data; (ii) generate contextual embeddings and knowledge graph linkages; (iii) enable immersive and passive observational data capture using augmented-and virtual-reality technologies; (iv) employ hybrid classical-quantum optimization to score, rank, or select individualized goals and intervention strategies; and (v) automatically generate auditable, evidence-linked applied behavior analysis reports with clinician-in-the-loop refinement.

### SUMMARY

This summary introduces a selection of concepts disclosed in greater detail throughout the specification and is not intended to limit the scope of the invention.

In some embodiments, the disclosed system aggregates multimodal applied behavior analysis data from structured repositories, unstructured clinical narratives, and sensor-derived behavioral observations obtained through immersive augmented-and virtual-reality interfaces or passive environmental sensing devices. The system normalizes the aggregated information into a unified schema and applies de-identification routines before downstream processing.

One or more processors execute natural language processing operations to tokenize text, perform linguistic annotation, and identify clinically relevant entities. A transformer-based architecture generates contextual embeddings representing relationships among symptoms, functions, goals, interventions, and outcomes. These embeddings may further be aligned with a knowledge graph that links entities across records, immersive interaction logs, and sensor-derived behavioral events.

In certain embodiments, the system incorporates an immersive reality interface that presents interactive therapeutic or assessment scenarios through an augmented-or virtual-reality display. One or more sensors associated with the immersive interface—such as optical, inertial, audio, or biometric sensors—capture the subject's behavioral responses during engagement. In alternative embodiments, the system operates in a passive observation mode that acquires behavioral data through environmental sensors, wearable devices, or ambient microphones and cameras.

A recommendation engine evaluates candidate goals, interventions, and measurement criteria by combining embeddings, graph features, historical outcome priors, and rule-based checks. In some embodiments, a hybrid classical-quantum optimization routine evaluates the candidate set under configured constraints to select or rank individualized goals, intervention steps, or program recommendations. The quantum component may implement a quadratic unconstrained binary optimization formulation or other quantum-compatible model to accelerate or improve selection performance for large or combinatorial decision spaces.

The system populates a report template with individualized goal statements, baseline descriptions, intervention procedures, and measurement systems, producing a machine-readable representation and a human-readable draft. A user interface presents the draft to a clinician, enables inline editing, and records each edit as a structured feedback signal that may be used to refine subsequent processing steps. Progress reports may be automatically generated at scheduled intervals by re-evaluating the most recent data and updating sections of the report accordingly.

These embodiments, along with others described herein, provide an integrated platform for multimodal behavioral-health analysis, automated reasoning across structured and unstructured clinical data, immersive and passive behavioral observation, and hybrid classical-quantum optimization to support individualized applied behavior analysis reporting and intervention planning.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram of a computing environment that may execute the disclosed modules and workflows, according to some embodiments;

FIG. 2 illustrates an application program and modules in communication with the computing system, including an AI module, ABA module, communication module, database engine, user module, and display module, according to some embodiments;

FIG. 3 depicts an operational dataflow arrangement in which a display module interacts with an artificial intelligence module, a virtual-reality interface module, a database engine, and an optimization subsystem to coordinate user interaction, immersive behavioral data capture, optimization processing, and persistence, according to some embodiments.

FIG. 4 depicts a pipeline in which the AI module, ABA module, communication module, database engine, and user interface operate together to generate, persist, and present applied behavior analysis content, according to some embodiments;

FIG. 5 depicts a governance and user interaction workflow that operates alongside the application program to control how content generated by the AI module and ABA module is exposed, audited, and improved through iterative use, according to some embodiments

FIG. 6 illustrates an immersive augmented-or virtual-reality behavioral capture subsystem, including one or more sensors integrated with an AR/VR device and configured to acquire behavioral signals during user engagement with interactive content, according to some embodiments.

FIG. 7 illustrates a passive environmental sensing subsystem that acquires behavioral data without requiring an immersive device, including ambient cameras, microphones, wearable devices, or other environmental sensors positioned within a therapy or naturalistic setting, according to some embodiments.

FIG. 8 illustrates a constrained decision-path selection subsystem in which candidate goals or interventions are converted into an optimization model, evaluated using quantum, quantum simulated, or quantum-inspired solvers, verified against policy constraints, and persisted as reproducible audit artifacts prior to downstream report generation, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The disclosed system may operate on a computing environment that includes one or more processors, system memory, non-transitory storage, and network interfaces that couple to data sources such as electronic health records, case notes, assessment instruments, and curated case studies. The processors may execute instructions stored in memory to coordinate data ingestion, privacy handling, natural language processing, representation learning, graph construction, recommendation scoring, template population, user interaction, and persistence. Each operation may be executed on a single machine or across a distributed cluster, and components may communicate through message queues or service calls over a secure network.

A data ingestion and normalization module may retrieve structured and unstructured content from local and remote repositories. The module may support connectors that query electronic health record systems through application programming interfaces, import delimited files and spreadsheets, and crawl document repositories that contain clinician notes or case studies. The module may assign a source identifier to each record, compute checksums for de duplication, and normalize timestamps to a common time zone. A schema mapping stage may translate heterogeneous fields into a shared schema for applied behavior analysis that includes entities such as client profile attributes, setting, antecedent, behavior, consequence, assessment scores, intervention steps, and outcome measures. The module may store normalized artifacts as versioned JSON documents or as rows in a relational database and may attach provenance metadata so downstream modules can trace each field to an origin.

A privacy and compliance module may remove or obfuscate personally identifiable information before any learning or modeling occurs. The module may apply pattern based detectors for telephone numbers, social security numbers, and dates of birth, and may use named entity recognition to flag likely names, addresses, and facility identifiers. For each detected span the module may write an annotation with an offset and a label, then may replace the text with a reversible token or with an irrecoverable placeholder depending on a configured policy. The module may maintain a de identification log stored in an access controlled vault so audit users can verify that a given record was processed. This module may also enforce access controls by redacting fields at query time based on a user's role and may log all data access events for later compliance review.

An NLP processing module may convert the de identified text into structured tokens and linguistic annotations that support downstream embedding and retrieval. The module may perform sentence segmentation, tokenization, part of speech tagging, and syntactic dependency parsing using a model trained on clinical or technical corpora. Lemmatization or stemming may reduce inflected forms to a canonical form, and a domain tuned named entity recognizer may label spans associated with observations, target behaviors, functions, assessments, interventions, and outcomes. The module may compute sentence level and document level summaries that preserve section boundaries such as intake, assessment, and session notes. Intermediate outputs may be stored in a document store so later stages can be re-run without revisiting upstream systems.

An embedding generation module may produce numerical representations that capture semantic relationships among symptoms, functions, goals, interventions, and outcomes. The module may host a transformer model that accepts tokenized text together with auxiliary features from the normalized schema. The model may output contextual embeddings at token level and pooled embeddings at sentence or document level. The module may project embeddings into a fixed dimensional space suitable for approximate nearest neighbor search and may index them with a vector database that supports cosine or dot product similarity. The module may augment embedding creation with domain prompts or adapter layers so that text about functional relations or skill acquisition programs maps to consistent neighborhoods.

A knowledge graph module may link entities across records to form a machine readable scaffold that supports reasoning and report population. The module may define node types for clients, settings, behaviors, assessments, goals, interventions, and outcomes, and edge types such as observed in, targeted by, precedes, co-occurs with, and achieves. Nodes may carry attributes including temporal validity windows, confidence scores, and provenance references to the source document and character offsets. The module may create edges by aligning named entities and by querying the vector index for semantically similar passages that mention compatible entities. A graph database may store the topology and may expose query patterns that retrieve, for example, interventions historically associated with a functionally similar behavior profile or goals that follow from a given assessment.

A recommendation and template population module may compute individualized goals and intervention guidance and may write those outputs into a selected report template. The module may accept as inputs a case context that includes the graph neighborhood around a client, the most similar historical cases retrieved from the vector index, and any structured assessment scores. A scoring routine may rank candidate goals by combining similarity scores from embeddings, support counts from graph edges, and rule based checks that enforce clinical sequencing. The module may select a template from a catalog keyed by payor, jurisdiction, or program type and may bind template fields to variables such as baseline level, goal statement, measurement criteria, intervention steps, and progress monitoring plan. When the module populates free text sections it may call a constrained generation routine that conditions on retrieved passages and emits text that references the underlying data through inline citations or field identifiers. The module may output a machine readable representation of the report and a human readable rendering for review.

A bias monitoring module may evaluate whether recommendation outputs exhibit disparities across protected or context specific attributes. The module may compute group metrics such as selection rate and score distributions by attribute and may compare them to configured thresholds. When the module detects a disparity it may adjust the recommendation score through calibrated reweighting, modify retrieval to balance neighborhood composition, or prompt the system to request additional case specific inputs. The module may persist audit artifacts that describe metric values, configuration states, and the exact data slices evaluated so that a reviewer can reproduce a decision path.

A user interface and feedback module may present proposed goals and interventions to a clinician and may collect edits that improve future computations. The interface may render the selected template with populated fields and may allow inline editing of text, toggling of recommended items, and entry of rationales. The module may record each edit as a structured event describing the original value, the edited value, and the justification. A background trainer may use these events as supervised signals to fine tune the embedding projection or the constrained generation routine. The interface may also expose search and filter controls over the knowledge graph and the vector index so users can inspect the evidence supporting each populated field.

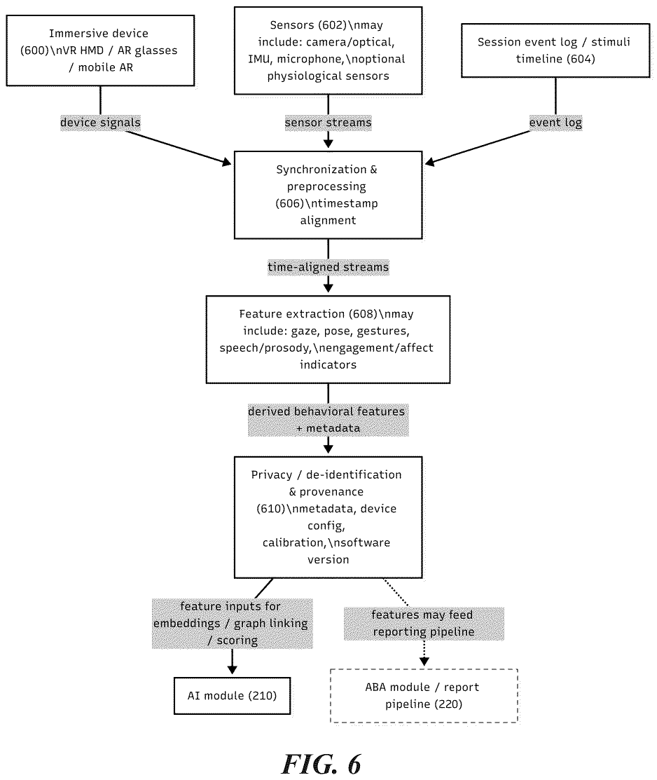

Immersive AR/VR behavioral capture subsystem (FIG. 6). In some embodiments, the disclosed system includes an immersive reality interface configured to present interactive content to a subject and to capture behavioral responses during engagement. The immersive reality interface may comprise a virtual reality head-mounted display, augmented reality glasses, or an augmented reality display provided by a tablet or mobile device. One or more sensors associated with the immersive reality interface capture behavioral observations in real time, including but not limited to eye gaze direction, eye movement, facial expressions, head and body motion, hand or controller interactions, reaction time, and vocalizations. By way of example, the immersive device may include one or more optical sensors (e.g., cameras), inertial sensors (e.g., accelerometers and gyroscopes), one or more microphones, and, in certain embodiments, physiological sensors (e.g., heart rate or skin conductance sensors).

In operation, the immersive reality interface generates session event logs that identify presented stimuli, task phases, prompts, and reinforcement events. The sensor observations are temporally aligned to the session event logs using timestamps, frame indices, or other synchronization markers. A preprocessing routine converts the raw sensor signals into feature representations suitable for downstream analysis, including gaze fixation metrics, head pose trajectories, gesture classifications, speech or prosody features, and derived engagement or affect indicators. The resulting time-series features and event-aligned summaries are ingested as additional inputs to the AI module for embedding generation, knowledge graph linking, recommendation scoring, and report population. The system may store the immersive session artifacts together with provenance metadata that identifies the device type, sensor configurations, calibration state, and software version used during capture.

In some embodiments, the immersive reality interface is integrated with the display module such that proposed goals, prompts, or intervention elements can be rendered within the immersive experience. The system may adapt the interactive content in real time based on detected behavioral responses, including adjusting stimulus difficulty, changing reinforcement schedules, or selecting alternate training scenarios, while recording the basis for each adaptation as structured events that remain auditable and reproducible.

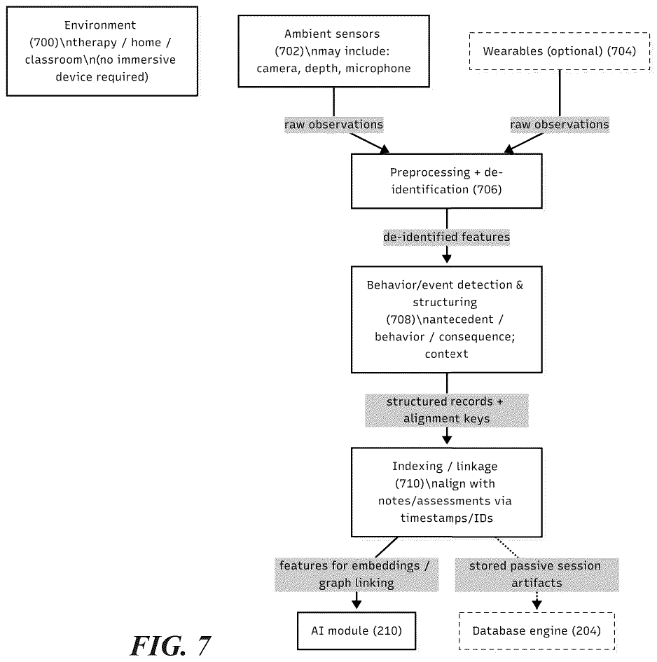

Passive environmental sensing subsystem (FIG. 7). In some embodiments, the system operates in a passive observation mode that captures behavioral data without requiring the subject to wear an AR/VR device. In this mode, the system may acquire observations through one or more ambient cameras, depth sensors, microphones, and/or wearable devices positioned in a therapy setting, home setting, classroom setting, or other naturalistic environment. Wearable devices may include, for example, wrist-worn sensors that capture motion and physiological signals. The passive observations may include body posture, locomotion, interaction patterns, facial expressions, vocalizations, and physiological responses. Immersive capture and passive environmental sensing are alternative, co-equal mechanisms for behavioral data acquisition, and either may be employed independently or in combination within the disclosed systems.

The passive observation mode may use the same preprocessing and privacy handling routines described herein. For example, visual or audio observations may be transformed into deidentified feature representations by detecting and removing personally identifiable information prior to storage or model processing. The system may create structured records representing detected behaviors, antecedents, consequences, or relevant context, and may align these records with session notes, assessment scores, and other structured data using timestamps or session identifiers. The resulting passive-sensing features may be embedded, indexed, and linked within the knowledge graph so that downstream recommendation and template population routines can incorporate both immersive-session observations and passive real-world observations within a unified case context.

In some embodiments, the system selects between immersive capture and passive capture based on subject tolerance, clinical goals, device availability, or configured policies. The system may also operate in a hybrid configuration in which passive sensing continues while an immersive session is conducted, thereby enabling cross-validation between immersive interaction events and environmental observations.

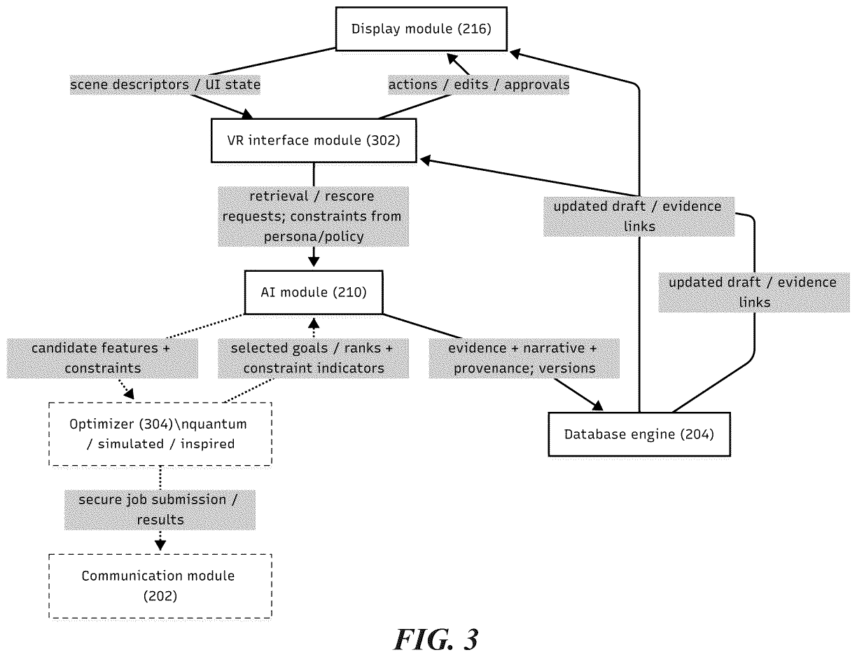

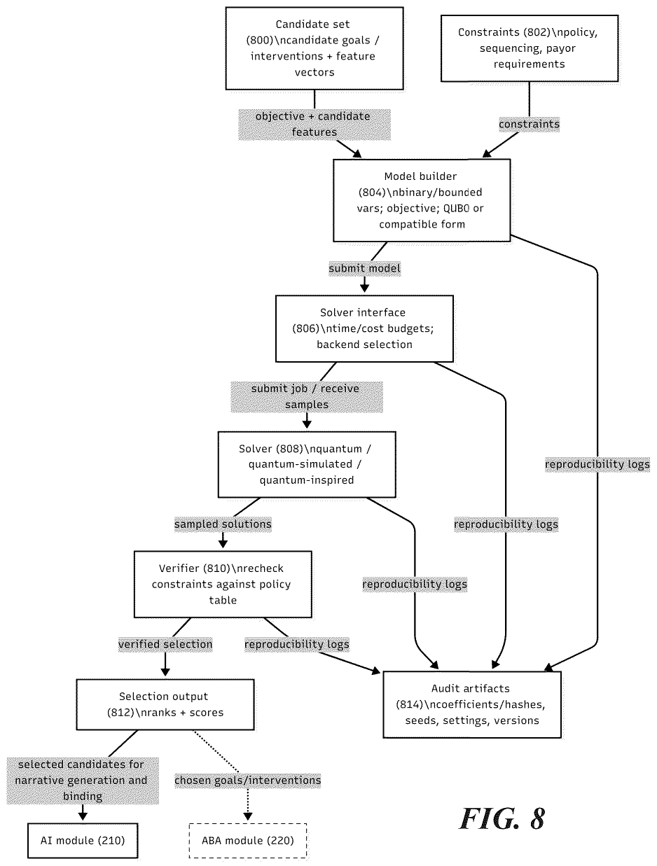

Hybrid classical-quantum optimization subsystem (FIG. 8). FIG. 8 illustrates the internal stages of a constrained decision-path selection subsystem that may be invoked by the operational dataflow shown in FIG. 3. In some embodiments, the system includes an optimization routine configured to select, rank, or allocate candidate goals, intervention steps, prompts, or program recommendations under constraints. The candidate set may be generated by the AI module and ABA module using embedding similarity, graph support signals, assessment scores, historical outcome priors, and rule-based checks. The system may then evaluate the candidate set under constraints such as session duration limits, prerequisite relationships, payor documentation requirements, clinical sequencing policies, or coverage requirements across skill domains.

In some embodiments, the optimization routine is implemented as a hybrid classical- quantum workflow. A classical preprocessing stage converts the candidate set and associated features into an optimization model, including binary or bounded variables and an objective function. The model may be expressed, by way of example, as a quadratic unconstrained binary optimization (QUBO) formulation or other quantum-compatible representation. A quantum computing resource executes at least a portion of the optimization, such as sampling candidate solutions or approximating an optimum under the objective function. The quantum computing resource may comprise a gate-based quantum processor, a quantum annealer, or a quantum inspired solver that follows the same model structure. A verification stage evaluates candidate solutions against the constraints and produces an optimized selection output that is supplied to the ABA module for template population and report generation.

The system persists the optimization inputs and outputs as audit artifacts, including the model coefficients or hashes thereof, constraint configurations, solver settings, and the selected goal or intervention set. These records enable a reviewer to reproduce the selection process and to trace populated report fields to the candidate features, evidence items, and constraint checks that influenced the optimization outcome.

The disclosed system may use a storage and indexing layer that persists normalized records, annotations, embeddings, graph structures, recommendation outputs, and reports. A relational database may store normalized tables, a document store may hold tokenized text with offsets and linguistic labels, a vector index may support fast nearest neighbor search over embeddings, and a graph database may maintain nodes and edges with versioning. Each store may maintain referential links through stable identifiers so that a report field can trace to a specific embedding, graph edge, and source sentence. A job scheduler may coordinate periodic refresh of embeddings and graph links when new data arrives or when a model is updated.

In one method of operation the processors may ingest new case notes and assessment results, assign them to a client profile, and run the privacy module to de identify the content. In embodiments employing immersive or passive sensing, the processors may also ingest behavioral observations captured via an AR/VR interface and/or environmental sensors, de-identify or obfuscate personally identifiable information within such observations, and generate time-aligned feature representations suitable for downstream embedding and graph linkage.

The NLP module may annotate the text and write token level labels. The embedding module may encode the text and insert vectors into the index. The knowledge graph module may align entities, update nodes and edges, and recalculate neighborhood embeddings. The recommendation module may select a template based on context, retrieve similar historical cases, score candidate goals and interventions, and populate the template fields. In embodiments employing hybrid classical-quantum optimization, the processors may construct an optimization model over the candidate set and invoke a quantum or quantum-inspired solver to select or rank individualized goals or intervention elements under configured constraints prior to populating the template fields.

The bias monitoring module may evaluate computed scores and adjust them when disparity thresholds are exceeded. The user interface may display the draft report and accept edits that the system stores as feedback signals.

The processors may generate progress reports at scheduled intervals by re-running the retrieval and scoring stages against the latest data. A diff routine may compare current outputs to previous reports and may mark updated sections for user attention.

When a user approves a report, the system may store a locked version with a signature and may export a formatted document according to external submission requirements. The system may also expose an application programming interface that allows partner applications to request a draft report given a case identifier and to retrieve structured outputs for downstream analytics.

Hardware deployment may vary. A single tenant installation may run all modules inside an enterprise network with access to on-premises databases. A multi-tenant installation may deploy containerized services on a cloud platform with network security groups that isolate tenants. The transformer model and vector index may run on hosts with graphics processing units for acceleration. Logs and metrics from each module may flow to a monitoring service that tracks latency, error rates, and resource use. Configuration files may define model versions, schema mappings, template catalogs, and bias thresholds, and may be version controlled so that changes are auditable.

Persons of ordinary skill in software engineering may implement the modules using common frameworks. For example, the NLP module may use a production ready library to perform tokenization and tagging, the embedding module may use a transformer checkpoint adapted with domain specific adapters, the vector index may use an approximate nearest neighbor library, the graph database may use a query language that supports path patterns, and the user interface may use a web framework that renders forms with validation and role based access controls. The modules may communicate through well-defined service contracts and may serialize data as JSON with explicit schemas for interoperability. In embodiments that incorporate immersive sensing or hybrid optimization, the frameworks may further include real-time data ingestion pipelines, hardware-accelerated computation on GPUs or other accelerators, and interfaces to quantum or quantum-inspired computing resources, while preserving modularity and interoperability across system components.

The disclosed system may be realized in several claim-supported embodiments. In a method embodiment the processors perform ingestion, privacy handling, NLP, embedding generation, graph construction, recommendation scoring, template population, bias evaluation, and report output. In a system embodiment the memory stores instructions that configure the processors to provide the described modules. In a computer readable medium embodiment the instructions cause a device to execute the same operations when loaded into memory and run by the processors. These embodiments may further incorporate immersive or passive behavioral sensing subsystems and hybrid classical-quantum optimization routines as described herein.

Various implementations of the invention involve the technical field of computer implemented applied behavior analysis including aggregating and normalizing, by one or more processors, applied behavior analysis data from structured and unstructured sources comprising at least electronic health records, case studies, clinical assessments, and user inputs; anonymizing, by the one or more processors, personally identifiable information contained in the applied behavior analysis data; processing, by the one or more processors executing an artificial intelligence model, the anonymized applied behavior analysis data using natural language processing to perform tokenization, lemmatization or stemming, part-of-speech tagging, and named entity recognition; generating, by the one or more processors, contextual embeddings using a transformer-based architecture to derive semantic relationships among symptoms, interventions, goals, and outcomes in an applied behavior analysis domain; detecting, by the one or more processors, bias in model outputs and adjusting a recommendation score responsive to the detected bias; selecting, by the one or more processors, a report template and auto-populating fields of the report template with individualized goals, interventions, and guidance based on the contextual embeddings; and outputting, by the one or more processors, a personalized applied behavior analysis report comprising an individualized intervention and behavior intervention guide, and are therefore necessarily rooted in computer technology. For example, the aforementioned steps are inherently computer-based and cannot be performed in the human mind. The present invention amounts to more than merely implementing the generic computer as a tool to gather, analyze, and output data because the steps of the present method, system, or product improve the computer implemented applied behavior analysis by providing a technical pipeline that ingests structured records and unstructured text from clinical sources, de identifies personally identifiable information, applies natural language processing, and generates transformer based contextual embeddings that link symptoms, goals, interventions, and outcomes across datasets, which enables automated template selection and auto population of individualized ABA reports and ongoing progress reports. The system further constructs and queries a knowledge graph, monitors outputs for bias with fairness aware algorithms, and incorporates clinician edits through a feedback loop that fine tunes subsequent recommendations. These mechanisms address data heterogeneity, privacy handling, and cross record reasoning that conventional tools treat separately by providing machine executable steps that transform and fuse large volumes of healthcare text and signals into structured, auditable report content. In eligibility terms, the claimed embodiments recite specific processing on a computer system and non-transitory media rather than a method of organizing human activity, because the operations require automated de identification, model based embedding generation, graph construction, vector retrieval, bias correction, and real time interface rendering at scales and speeds that cannot be performed in the human mind or with pen and paper and are necessarily rooted in computer technology. In particular, the speed at which the steps of the present invention occur to effectuate the disclosed method, system, or product would involve large-scale, continuous wireless communication of such data. That is, the steps of the present method, system, or product are impossible to accomplish on pen and paper, cannot be accomplished as a method of organizing human activity, and amount to significantly more than merely gathering, analyzing, and outputting data.

Implementations of the disclosed system execute one or more artificial intelligence models on specific computer hardware using compiled machine learning libraries that invoke tensor operations on CPUs and GPUs. The processors read de identified clinical text and structured records from encrypted storage, segment the text into tokens with byte offsets, and compute transformer-based contextual embeddings that map millions of tokens per minute into high dimensional vectors. A vector index receives the embeddings and builds approximate nearest neighbor structures in main memory to support sub second retrieval across large corpora. A knowledge graph store then materializes nodes and edges with temporal validity and provenance, while a bias monitoring routine streams group metrics to a metrics service and applies calibrated reweighting to recommendation scores. These steps alter data formats, memory layouts, and storage states at every stage, including token sequences, tensor values, vector indices, and graph topologies, and they run under back pressure-controlled pipelines that coordinate GPUs, system memory, and disk I/O.

The disclosed system integrates the models into a concrete computing workflow that trains on curated corpora, performs inference under latency budgets, and writes deterministic outputs into report templates through bounded decoding and schema binding. The processing is not practically performable in the human mind or using pen and paper because it requires simultaneous execution of billions of floating-point operations, streaming de identification over gigabytes of text, vector similarity search over millions of embeddings, and graph updates that enforce referential integrity and versioning. The claimed embodiments recite a method performed by processors, a machine comprising processors and non-transitory memory storing executable instructions, and a computer readable medium with instructions that, when executed, cause these hardware level transformations. The operations apply the models to specific input signals from electronic health records, case notes, and assessments, transform those signals into embeddings and graph structures, and drive automated template population with audit logs and access controls, which represents a practical application that improves computer based clinical documentation by reducing latency, increasing consistency, and enabling traceable, reproducible outputs rather than a mere method of organizing human activity.

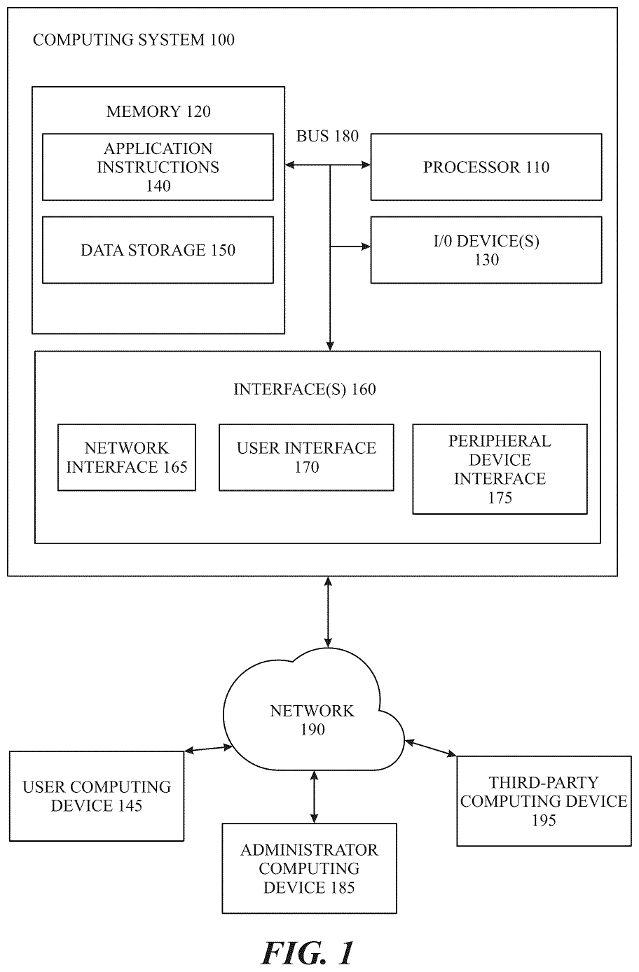

FIG. 1 illustrates an example of a computing system 100 that may provide the execution environment for implementing the processes and methods described herein. The computing system 100 may take various forms depending on deployment context, including but not limited to: a desktop or laptop computer, a tablet or smartphone, a server in a data center, a network appliance, a mainframe computer, a workstation, or a cloud-hosted virtual machine. In some embodiments, the computing system 100 may correspond to a distributed computing environment, such as a cluster of servers executing containerized workloads (e.g., Docker, Kubernetes), or an edge device integrated into Internet of Things (IoT) environments. In other embodiments, the computing system 100 may be embedded in another device, such as a vehicle infotainment unit, a medical diagnostic machine, an industrial robot controller, or a wearable computing device.

The computing system 100 includes one or more processors 110 operably coupled to a memory 120 via a system bus 180. The processor 110 may be implemented as a general-purpose central processing unit (CPU), a graphics processing unit (GPU), a tensor processing unit (TPU), a digital signal processor (DSP), or any combination thereof. In some embodiments, the processor 110 may comprise an application-specific integrated circuit (ASIC) optimized for a particular workload, a field-programmable gate array (FPGA), or, in advanced implementations, a quantum or neuromorphic processor. The processor 110 may include single-core, multi-core, or many-core configurations and may support hardware virtualization, multithreading, or parallel execution environments to optimize system performance.

The memory 120 may include volatile memory, nonvolatile memory, or a combination thereof. Volatile memory may include system RAM, cache memory, or high-bandwidth memory (HBM). Nonvolatile memory may include flash storage, solid-state drives (SSD), magnetic hard disk drives (HDD), optical storage devices, or persistent memory technologies such as Intel Optane. The memory 120 stores application instructions 140 for carrying out the functionalities described herein and data storage 150 for maintaining information related to system operations. The application instructions 140 may include code written in languages such as C, C++, Java, Python, Go, Rust, or JavaScript, as well as machine learning models trained using frameworks such as TensorFlow or PyTorch. The data storage 150 may contain structured information such as relational database records, unstructured data such as text or images, or real-time telemetry streams. In cloud-based embodiments, the memory 120 may represent scalable storage resources provisioned on-demand through Infrastructure-as-a-Service (IaaS) providers.

The computing system 100 may also include one or more input/output (I/O) devices 130. These devices may encompass visual output devices such as monitors, head-mounted displays, augmented reality (AR) glasses, or projectors; input devices such as keyboards, mice, touchscreens, styluses, or game controllers; and sensor devices such as microphones, cameras, depth sensors, biometric scanners, or environmental sensors. In industrial or medical environments, the I/O devices 130 may include robotic actuators, infusion pumps, or diagnostic imaging scanners. In vehicular environments, the I/O devices 130 may include in-cabin displays, steering sensors, and connected infotainment systems.

The computing system 100 further comprises one or more interfaces 160 that enable communication with other systems, users, or peripheral components. The network interface 165 allows the computing system 100 to exchange data with external systems across a network 190 using wired or wireless protocols. Example communication standards include Ethernet, Wi-Fi, Bluetooth, 5G, Long-Term Evolution (LTE), satellite communication, or emerging protocols such as Wi-Fi 7 or ultra-wideband (UWB). In some embodiments, the network interface 165 supports secure protocols such as HTTPS, TLS, or VPN tunneling to ensure authenticated and encrypted data transfer. The user interface 170 may include APIs, graphical user interfaces (GUIs), command-line interfaces (CLIs), or natural language interfaces enabled through speech recognition or chatbot systems. The peripheral device interface 175 enables connectivity with external hardware such as printers, external storage arrays, or specialized scientific equipment.

The network 190 represents any communication infrastructure capable of facilitating data exchange between computing entities. In some embodiments, the network 190 corresponds to a local area network (LAN) within a home or enterprise environment. In other embodiments, the network 190 may be a wide area network (WAN), a metropolitan area network (MAN), a peer-to-peer (P2P) communication mesh, or the global Internet. The network 190 may employ cloud orchestration layers, software-defined networking (SDN), or edge computing gateways. In high security applications, the network 190 may implement firewalls, intrusion detection systems, or zero-trust architectures to protect transmitted data.

The computing system 100 is illustrated as being in communication with multiple external devices, including a user computing device 145, an administrator computing device 185, and a third-party computing device 195. The user computing device 145 may be a smartphone, tablet, laptop, or smart appliance configured to execute client-side applications or interact with system services. The administrator computing device 185 may be a workstation or remote management console configured to perform oversight functions such as monitoring, auditing, updating, or troubleshooting. The third-party computing device 195 may represent a partner system, vendor service, or external application interface that exchanges data with the computing system 100 via secure APIs. In cloud or SaaS embodiments, these devices may also include external microservices, data warehouses, or federated learning nodes.

In some embodiments, the computing system 100 may be deployed in a client-server model, where the computing system 100 acts as a backend server managing requests from client devices. In other embodiments, the computing system 100 may function within a cloud-native environment, operating as a microservice within a container orchestration platform. In edge deployments, the computing system 100 may be optimized for low-latency local processing, while synchronizing with centralized cloud infrastructure for data persistence and global coordination.

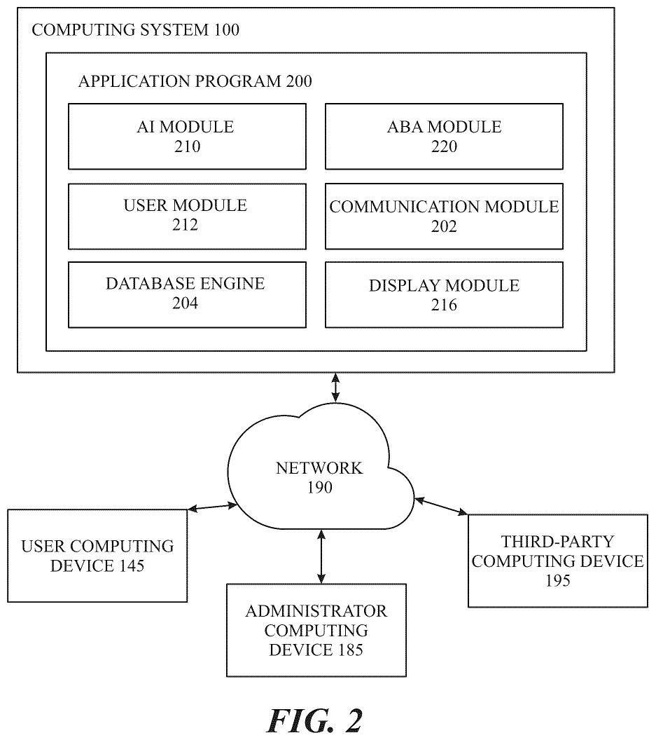

FIG. 2 illustrates an example application architecture for the application program 200 operated by the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of an AI module 210, an ABA module 220, a communication module 202, a database engine 204, a user module 212, and a display module 216.

FIG. 2 illustrates a computing system 100 executing an application program 200 comprising coordinated software components configured to generate applied behavior analysis reports and individualized guidance. The processors of computing system 100 load application program 200 from non-transitory memory and execute instructions that pass data and control signals among the depicted modules. Each module may run as a process or container and may communicate over secure inter-process channels. The arrangement shown in FIG. 2 groups functional capabilities into six cooperating modules labeled AI module 210, ABA module 220, user module 212, communication module 202, database engine 204, and display module 216.

Communication module 202 provides network connectivity and external system integration for application program 200. The module maintains client and server endpoints, negotiates transport security, and exposes application programming interfaces that allow electronic health record systems, assessment platforms, and case repositories to exchange data with computing system 100. Communication module 202 manages authentication tokens, session lifecycles, retry policies, and rate limiting so that upstream data sources can stream structured records and unstructured documents reliably. The module writes incoming payloads to a staging area, computes checksums to detect duplicates, and publishes ingest events that trigger downstream processing by AI module 210 and database engine 204.

Database engine 204 persists and indexes all structured and derived artifacts used by application program 200. The engine may include a relational store for normalized records, a document store for de-identified text with token offsets and annotations, a vector index for contextual embeddings, and a graph store for entities and relationships that link client attributes, observed behaviors, assessments, goals, interventions, and outcomes. Database engine 204 enforces schemas, foreign key constraints, and versioning, and it maintains provenance identifiers that bind each stored element to a data source and processing step. The engine services queries from AI module 210 and ABA module 220 by optimizing execution plans, managing cache lifetimes, and streaming results with back pressure to match consumer throughput.

AI module 210 operates as a set of cooperating services that transform de-identified clinical text and structured records into machine-readable representations, retrieve semantically relevant context at scale, generate bounded narrative outputs and score recommendations used for template population. The module loads one or more transformer-based models that have been adapted to applied behavior analysis terminology using domain prompts or parameter-efficient adapters. The processors of computing system 100 execute the models through optimized tensor libraries so the module can encode millions of tokens into vectors within latency budgets required for interactive use. AI module 210 exposes callable endpoints so other components can request tokenization, embedding, retrieval, evidence aggregation, text generation, and bias evaluation as discrete operations or as an orchestrated workflow.

Within AI module 210, a text processing service accepts UTF-8 text and offset maps from upstream privacy handling and performs sentence segmentation, tokenization, part-of-speech tagging, and dependency parsing. The service attaches stable token identifiers, character spans, and linguistic features that downstream routines use to preserve traceability between generated outputs and source text. A domain entity recognizer labels spans for antecedents, behaviors, consequences, assessment names, skill targets, measurement criteria, and outcomes. The service persists these annotations as sidecar documents keyed by record identifiers so that repeated calls do not reprocess unchanged inputs.

An embedding service feeds token sequences and optional structured features into a transformer backbone to compute contextual embeddings. It produces token-level vectors for fine grained alignment as well as pooled vectors at sentence and document scopes for retrieval. The service projects the vectors into a fixed-dimensional space and inserts them into a vector index that supports cosine or dot-product similarity with approximate nearest neighbor search. The index returns identifiers and distances for the top-k matches, and the service bundles those results with provenance metadata and confidence values. To control drift, the service versions the projection parameters and maintains per-version namespaces in the index so historical reports can be reproduced exactly.

A knowledge linking service aligns recognized entities across records and builds or updates graph elements managed by database engine 204. The service creates nodes for client attributes, settings, target behaviors, assessment findings, goals, interventions, and outcomes, and it instantiates edges such as observed-in, targeted-by, precedes, and achieves with temporal validity intervals. The service reconciles near duplicates by comparing token-level embeddings around entity spans and by applying rule checks on codes and dates. It assigns provenance to every node and edge so the system can highlight source sentences in the display module 216 and so auditors can re-run the same linking steps against frozen inputs.

A retrieval and evidence synthesis service accepts a case context from ABA module 220 and queries both the vector index and the knowledge graph to assemble a working set of semantically similar passages and structurally related events. The service merges these results using learned or configured weights that trade off lexical similarity, embedding distance, graph path length, and recency. It formats the working set as conditioned context, with each evidence item carrying a pointer to its source and a content hash. Downstream generation and scoring routines consume this conditioned context so the system can cite or preview the exact evidence supporting each populated field.

A constrained generation service produces narrative text for goal statements, intervention steps, and rationales under deterministic controls. The service uses the transformer decoder in a retrieval-augmented configuration that conditions on the synthesized evidence while applying decoding constraints such as allowed vocabularies for measurement criteria, maximum token budgets per section, and schema-bound placeholders for client-specific variables. The service rejects tokens that would reveal personally identifiable information by consulting a denylist derived from the de-identification log, and it inserts inline provenance markers that reference evidence identifiers rather than copying raw text. The service emits both the human-readable text and a machine-readable structure that binds each sentence to the evidence items and template fields it supports.

A recommendation scoring service ranks candidate goals and interventions for inclusion in a draft report. The service computes a composite score that aggregates embedding similarity, graph support counts, outcome priors derived from historical cohorts, and rule checks that model clinical sequencing. The service calibrates scores using isotonic regression or temperature scaling so thresholds correspond to stable acceptance rates across cases. It returns a ranked list with feature contributions so user module 212 and display module 216 can present explanations alongside each recommendation.

A bias monitoring service evaluates outputs for disparities across configured attributes such as age ranges or service settings. The service streams batched scores and selections to an internal metrics store, computes group metrics, and compares them to policy thresholds. When a disparity exceeds a threshold, the service can reweight candidates, broaden retrieval neighborhoods, or flag the draft for additional user review. The service persists metric snapshots and configuration hashes so reviewers can reproduce the exact evaluation that led to a mitigation.

AI module 210 maintains a feedback learning loop that converts clinician edits and accept/reject actions into supervised signals. The module records each change as a tuple containing the original value, the edited value, the evidence set, and the surrounding context. A trainer process samples these tuples to fine tune adapter layers in the encoder and to adjust decoding constraints and scoring weights. The trainer writes new model artifacts and constraint profiles with semantic version numbers and only promotes them to production after offline replay confirms that previously approved reports remain stable.

Operationally, AI module 210 manages resources and security to sustain throughput and privacy. The module batches requests to maximize GPU utilization, enforces admission control to prevent overload, and shards vector indices by tenant or cohort to bound latency. It authenticates every call from other modules, permits only de-identified inputs, and redacts any decoded text that matches a sensitive pattern before returning results. Telemetry captures per-stage latency, token counts, retrieval distances, and decoding entropy so operators can diagnose regressions and so ABA module 220 can adapt its orchestration based on current capacity.

Through these cooperating services, AI module 210 receives normalized, de-identified inputs from communication module 202 and database engine 204, produces embeddings, graph links, evidence sets, narrative text, scores, and bias diagnostics, and returns deterministic artifacts that ABA module 220 binds into a report template. Each output carries identifiers and provenance so display module 216 can present transparent justifications and so database engine 204 can store immutable, auditable records suitable for later verification or regeneration.

ABA module 220 provides domain orchestration for applied behavior analysis workflows and operates as the layer that turns model outputs and clinical inputs into a complete, submission ready report package. The processors of computing system 100 load ABA module 220 as a service that maintains configuration for programs, payors, and jurisdictions, and exposes callable interfaces to AI module 210, database engine 204, user module 212, communication module 202, and display module 216. ABA module 220 maintains a stateful case context in memory that references the client profile, session history, assessment scores, graph neighborhoods, and embeddings required to assemble individualized goals, intervention steps, and measurement plans.

ABA module 220 implements a template manager that selects a report or progress note template and binds fields to data sources. The template manager may index a catalog keyed by payor, state, provider type, and document purpose, and it may encode each template as a schema with required sections, allowed vocabularies, and validation constraints. On a new case, the template manager queries database engine 204 for program and payor metadata, computes an eligibility rule set, and chooses a base template. Field binders in the template manager map each template field to a variable or generator, such as baseline metrics from normalized records, goal statements from AI module 210, or measurement criteria from configured outcome scales. The template manager persists the chosen template and binder map with a semantic version so a reviewer can reproduce the draft later.

ABA module 220 runs a goal and intervention synthesizer that converts evidence and embeddings into actionable plan elements. The synthesizer requests from AI module 210 a working set of similar cases, supporting passages, and candidate goal and intervention snippets tied to knowledge graph nodes. The synthesizer evaluates these candidates with domain rules that reflect sequencing conventions, prerequisite skills, and measurement compatibility. For example, when the synthesizer detects a discrete trial training context, it binds measurement criteria to event based counts, while a natural environment training context shifts measurement to time-sampled observations. The synthesizer assembles each plan element as a structured object containing a goal statement, present level, measurement system, prompts or cues, teaching procedures, and generalization strategies, with pointers to the evidence items that supported the selection.

ABA module 220 includes a scoring and constraint engine that ranks candidates and enforces compliance requirements. The engine computes a composite score for each goal and intervention by combining embedding similarity returned by AI module 210, support counts along graph paths, outcome priors from historical cohorts stored in database engine 204, and rule checks that ensure alignment with assessment findings. The engine calibrates scores against configured acceptance thresholds so the same numeric value yields consistent inclusion decisions across programs. The constraint subsystem validates that every required section is present, that measurement criteria use allowed scales and targets, that planned session frequencies fall within configured ranges, and that the plan contains cross-setting generalization when required. When any constraint fails, the engine emits a structured defect with a suggested remedy and routes the plan back through the synthesizer or prompts user module 212 to request additional inputs.

ABA module 220 operates a plan composer that assembles the validated elements into a machine-readable report object and a human-readable draft. The composer merges fixed template content with populated fields, inserts rationale sentences generated by AI module 210 under bounded decoding, and embeds provenance markers that reference evidence identifiers rather than raw text. The composer assigns persistent identifiers to each paragraph and field, writes a diff index against prior versions, and records authorship and timestamps so display module 216 can show change tracking and so database engine 204 can store immutable versions for audit.

ABA module 220 runs a progress scheduler that automates periodic updates. The scheduler registers triggers such as elapsed time since last report, new session uploads received through communication module 202, or attainment thresholds or milestones recorded in database engine 204. On a trigger, the scheduler requests refreshed embeddings, retrievals, and graph updates from AI module 210, re-evaluates goal attainment and trend direction, and regenerates only the affected sections while preserving approved content. The scheduler marks regenerated sections for user review in display module 216 and maintains links between superseded and current text so users can trace how a plan evolves.

ABA module 220 maintains a compliance and export adapter that prepares finalized documents for external submission. The adapter applies redaction rules based on user roles set by user module 212, resolves template-specific formatting, and builds export packages such as PDFs and machine-readable bundles defined by payor or registry specifications. Before export, the adapter runs a final validation sweep that checks section presence, reference integrity to evidence items, and signature blocks. The adapter then posts the package to external endpoints through communication module 202 and records delivery receipts and checksum hashes in database engine 204 for non-repudiation.

ABA module 220 closes the loop with a feedback collector that converts clinician actions into training and policy signals. When a clinician accepts, edits, or rejects a goal or intervention in display module 216, the collector records the original recommendation, the edit, the evidence set, the constraint checks that were active, and the final disposition. The collector streams these tuples to AI module 210 for adapter fine-tuning and to the scoring and constraint engine for weight updates. ABA module 220 promotes new policy versions only after replaying historical cases confirms that previously approved outputs remain stable and that measured error and latency budgets stay within configured bounds.

Through these cooperating subfunctions, ABA module 220 orchestrates template selection, goal and intervention synthesis, rule-based validation, versioned composition, scheduled updates, compliant export, and feedback capture. The module grounds every populated field in retrievable evidence and deterministic rules, coordinates tightly with AI module 210 for retrieval and generation, persists artifacts through database engine 204, communicates with external systems via communication module 202, and exposes an interactive experience through display module 216 under authentication and auditing managed by user module 212.

User module 212 manages identity, roles, and preferences for end users and administrators who interact with application program 200. The module authenticates users through the communication module 202, establishes role-based access controls that govern visibility of de identified versus re identified fields, and records granular audit events for compliance review. User module 212 captures clinician feedback on generated goals and narrative text by logging edits, toggles, and rationales as structured events. These events flow to AI module 210 as supervised signals for fine tuning and to ABA module 220 as constraints that influence future template population. The user module 212 also stores interface preferences and notification settings that display module 216 uses to tailor views and alerts.

Display module 216 renders graphical user interfaces that present data, recommendations, and reports produced by application program 200. The module reads the structured draft prepared by ABA module 220 and generates interactive forms that show populated fields next to evidence snippets retrieved through AI module 210. Display module 216 supports inline editing with validation against schema rules enforced by database engine 204, presents bias monitoring summaries and provenance links for transparency, and streams real-time status indicators for data ingestion and processing tasks reported by communication module 202. The module prepares export artifacts such as payor specific PDFs and machine readable bundles and transmits them through communication module 202 to external recipients when a user authorizes release.

The components of application program 200 operate as a pipeline when computing system 100 processes a case. Communication module 202 receives source records and posts ingest events. Database engine 204 writes normalized entries and exposes them to AI module 210 for annotation, embedding, and knowledge graph updates. ABA module 220 requests nearest neighbor retrieval and scoring from AI module 210, selects a template, and assembles a draft report. Display module 216 presents the draft for review, while user module 212 authenticates the reviewer, enforces field level permissions, and captures edits and approvals. Database engine 204 finalizes the report with signatures and version stamps, and communication module 202 delivers the finished output to designated systems. The architecture shown in FIG. 2 grounds each software function in a defined module that specifies what it is, what it does, and how it performs its subfunctions within computing system 100.

FIG. 3 depicts a dataflow arrangement in which display module 216 interacts with a VR interface module 302 that coordinates user interaction with downstream analytics and persistence services. Display module 216 renders draft and finalized content on a conventional display while streaming the same content as structured scene descriptors to VR interface module 302. The scene descriptors may include panel layouts, evidence snippets, selectable recommendations, and provenance links. VR interface module 302 translates controller events, gaze targets, and hand gestures into normalized actions such as approve, edit, request evidence, and rescore. The module maintains a bidirectional channel to display module 216 so that edits made in an immersive view appear in the conventional view without delay and so accessibility settings or presentation themes selected on the display carry into the immersive scene. FIG. 3 illustrates an end-to-end operational dataflow incorporating an optimization subsystem, rather than the internal structure of the optimization subsystem itself.

VR interface module 302 forwards analytic requests and edit events to AI module 210 and to communication module 202. When a user requests additional supporting passages or alternative goals inside the immersive view, VR interface module 302 sends a retrieval job to AI module 210 that identifies the active client context, the focused section of the report, and any constraints set by policy. For actions that fetch external records or notify collaborators, VR interface module 302 calls communication module 202, which authenticates against external systems, applies retry and rate control, and returns payloads to the VR session as normalized objects. The module coalesces rapid user inputs into batches so AI module 210 and communication module 202 receive steady workloads rather than bursts that could increase latency.

AI module 210 performs the natural language processing, embedding generation, retrieval, knowledge linking, bounded text generation, recommendation scoring, and bias monitoring described in the detailed description. In the FIG. 3 flow, AI module 210 also computes optimization variables that summarize candidate goals and interventions for a given case. For each candidate, the module emits a vector of features that may include embedding similarity to prior successful cases, graph support counts along clinically relevant paths, expected attainment time, workload impact, and compliance flags. AI module 210 supplies these feature vectors to a downstream optimizer as an intermediate artifact together with constraints derived from policy and user role selections captured by VR interface module 302.

A quantum optimizer 304 receives the candidate set and constraints and computes a selection or ordering that maximizes a defined objective such as expected therapeutic value subject to session frequency limits, prerequisite relationships, and payor documentation requirements. The optimizer may operate in quantum, quantum-simulated, or quantum-inspired modes. In a quantum mode the optimizer formats the objective and constraints as a quadratic unconstrained binary optimization instance, transmits the instance to a quantum processing service over a secure channel, and retrieves samples that approximate the optimum. In a simulated or quantum-inspired mode, the optimizer runs on the processors of computing system 100 using annealing or variational heuristics that follow the same objective structure. In all modes the optimizer logs the exact coefficients, seeds, and sampler settings so a reviewer can reproduce the decision offline.

Quantum optimizer 304 implements subfunctions that prepare, solve, and verify optimization problems before any selection propagates to the rest of the pipeline. A model builder converts candidate features into binary or bounded integer variables and encodes constraints such as mutual exclusivity, minimum coverage of skill domains, and upper bounds on weekly minutes. A sampler interface submits the model to the selected backend and enforces timeout and cost budgets. A verifier maps sampled solutions back to domain objects and rechecks every constraint against the authoritative policy table stored by database engine 204. If the verifier detects a violation it requests additional samples or relaxes secondary objectives under a configured policy that never permits constraint breaches. The internal stages illustrated in FIG. 8 are isolated for clarity and may be implemented independently of any user interface or sensing modality.

The verified solution returns to AI module 210 as a set of chosen goals and interventions with per item scores and rank positions. AI module 210 merges these selections with retrieved evidence and generates bounded narrative text for each chosen item. The module annotates every generated sentence with references to the evidence items, the optimization variables that influenced the selection, and the provenance of those variables. AI module 210 then emits a structured package that ABA module 220 can bind into a report template. In the FIG. 3 flow the package proceeds directly to database engine 204, while ABA module 220 may read it from storage when composing the draft.

Database engine 204 persists all artifacts created during the FIG. 3 sequence. The engine writes the feature vectors provided to quantum optimizer 304, the optimization model coefficients, the sampled solutions, the chosen set, and the generated narrative with full provenance. The engine maintains referential integrity across these records so that an auditor or a clinician can trace any populated field shown to the user back to the evidence item and to the exact optimization problem that influenced its inclusion. Database engine 204 also publishes change events that inform display module 216 and VR interface module 302 that new content is available for rendering.

Communication module 202 participates throughout the FIG. 3 sequence by brokering messages between internal services and external endpoints. For calls to a remote quantum processing service, communication module 202 signs requests, manages encryption of payloads, enforces per tenant quotas, and records receipts and checksums. For inbound clinical data triggered by a user action in VR interface module 302, the communication module 202 authenticates to electronic health record systems, normalizes the payload format, and stores the result in database engine 204 before signaling AI module 210 that new material is ready for embedding and graph updates. The module also handles notifications to collaborators by posting status updates and receiving acknowledgments that database engine 204 binds to the active report version.

Display module 216 closes the loop by presenting every new selection, narrative paragraph, and evidence link returned from database engine 204. In a conventional view the module highlights the role that quantum optimizer 304 played by showing selected items with their rank position and constraint satisfaction indicators. In the immersive view mediated by VR interface module 302 the same elements appear as layered panels that a user can expand to review supporting evidence or to invoke a rescore with modified preferences. Any accept, edit, or reject action that the user performs propagates back through VR interface module 302 to AI module 210 and, when relevant, triggers a fresh optimization cycle in quantum optimizer 304, after which database engine 204 stores a new version and the display updates to show the changes.

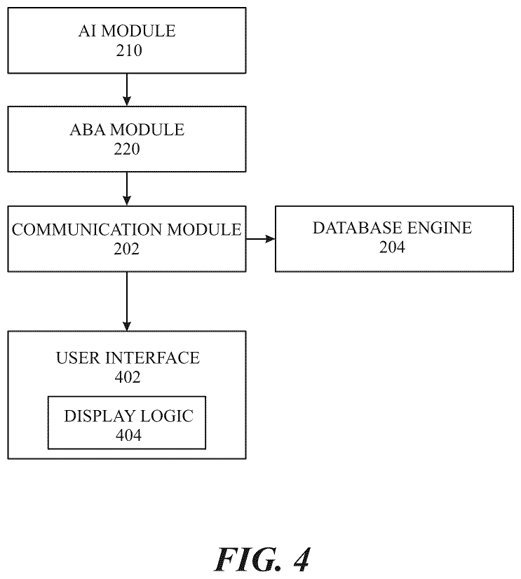

FIG. 4 depicts a pipeline in which AI module 210, ABA module 220, communication module 202, database engine 204, and user interface 402 with display logic 404 operate together to generate, persist, and present applied behavior analysis content. The flow shows AI module 210 producing machine representations and candidate narrative, ABA module 220 assembling and validating plan elements, communication module 202 brokering data exchange and persistence, database engine 204 storing artifacts with provenance, and user interface 402 rendering an interactive view through display logic 404. The processors of computing system 100 execute these components as cooperating services that exchange typed messages and identifiers so every field shown to a clinician traces to deterministic computations and stored evidence.

AI module 210 receives de-identified clinical text and structured records and performs tokenization, linguistic labeling, embedding generation, vector retrieval, knowledge linking, bounded text generation, recommendation scoring, and bias monitoring. The module emits artifacts that include token offset maps, contextual embeddings, graph node and edge definitions, ranked goal and intervention candidates with feature contributions, and narrative snippets produced under decoding constraints. Each artifact carries a stable identifier, a model version tag, and a content hash so downstream services can verify integrity and reproduce results. AI module 210 returns these artifacts to ABA module 220 as serialized payloads and also exposes endpoints that communication module 202 can call for asynchronous jobs.

ABA module 220 receives the AI outputs and applies applied behavior analysis domain logic to construct a draft plan. The module selects a report or progress template keyed by program and payor, binds template fields to variables and generators, evaluates candidate goals and interventions against clinical sequencing rules, and composes a structured report object that includes baseline measures, goal statements, measurement criteria, intervention steps, and progress monitoring plans. ABA module 220 validates section presence, reference integrity, and unit compatibility, and emits a versioned draft with a diff index against any prior version. The module forwards the draft and its binder map to communication module 202 for persistence and presentation.

Communication module 202 functions as a transport and coordination layer between compute services and storage while also handling external system exchange. The module authenticates callers, enforces rate limits, and assigns a correlation identifier to each transaction. On receipt of a draft package from ABA module 220, the module writes the payload to durable queues, persists metadata to a job ledger, and invokes database engine 204 using transactional calls so the entire draft, its evidence references, and its lineage either commit together or roll back together. When external records or notifications must be exchanged, the module negotiates secure connections, normalizes payloads to schemas used by the system, and records acknowledgments and checksums that become part of the provenance.

Database engine 204 provides persistence and indexing for normalized records, annotations, embeddings, graph structures, recommendation scores, drafts, and final reports. In the FIG. 4 flow the engine accepts inserts and updates from communication module 202 and stores them in coordinated layers that may include a relational store for normalized entities, a document store for annotated text with token offsets, a vector index for nearest neighbor search, and a graph store for nodes and edges linking client attributes to interventions and outcomes. The engine maintains foreign keys across these layers through stable identifiers produced by AI module 210 and ABA module 220, records semantic versions of models and templates, and exposes read optimized views that user interface 402 queries to render evidence aligned content.

User interface 402 presents interactive forms, evidence views, and status indicators to clinicians and administrators. The interface requests the latest draft and supporting artifacts from communication module 202, which proxies the requests to database engine 204 when necessary, and it renders the content using display logic 404. The interface accepts edits, approvals, rejections, and comments as structured events and posts them back through communication module 202 so they persist as feedback tuples and audit records. Role based access controls supplied by a user management service govern which fields appear re identified and which remain redacted at display time.

Display logic 404 implements the rendering and interaction subfunctions that turn stored artifacts into a coherent, navigable experience. The logic binds template fields to on screen widgets, enforces client side validation that mirrors the schema rules encoded by ABA module 220, and shows inline provenance indicators that reference the exact evidence identifiers and graph paths stored by database engine 204. When a user expands an evidence marker, display logic 404 fetches the supporting passage, the embedding distance, and the knowledge graph context and presents them without leaving the current section. The logic also renders difference views between versions by consulting the diff index emitted by ABA module 220 so a reviewer can see precisely which sentences changed between drafts.

The data path in FIG. 4 proceeds top down for computation and left to right for persistence. AI module 210 computes features and candidate narrative and passes them to ABA module 220, which assembles the draft and forwards it to communication module 202. Communication module 202 writes artifacts into database engine 204 under transactional control and returns references to user interface 402. User interface 402 retrieves the stored artifacts and display logic 404 renders a synchronized view, after which any user action travels back through communication module 202 to create new versions and to supply training and policy signals to the compute services.

Each component shown in FIG. 4 performs measurable functions using explicit data structures and protocols. AI module 210 transforms text and signals into embeddings, graph links, and generated text under bounded decoding. ABA module 220 translates model outputs and clinical rules into a validated, versioned report object. Communication module 202 orchestrates delivery, persistence, and external exchange while preserving atomicity and auditability. Database engine 204 maintains immutable records, indices, and lineage so every rendered field can be traced to its sources. User interface 402 with display logic 404 presents these artifacts, enforces client side constraints, and captures feedback that the system uses to refine subsequent computations. The arrangement enables reproducible, auditable report generation with clear separation of computation, orchestration, transport, storage, and presentation.

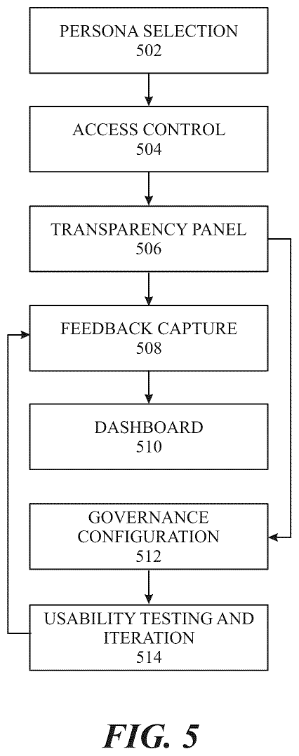

FIG. 5 depicts a governance and user interaction workflow that operates alongside application program 200 to control how content generated by AI module 210 and ABA module 220 is exposed, audited, and improved through iterative use. The flow begins with persona selection 502, proceeds through access control 504 and a transparency panel 506, captures clinician and administrator input at feedback capture 508, summarizes status in a dashboard 510, applies policy at governance configuration 512, and drives continuous improvement through usability testing and iteration 514. Arrows indicate closed loop operation in which later stages publish constraints and signals that modify earlier stages during subsequent sessions.

Persona selection 502 identifies the role, objectives, and context for a current session and provides parameters to user module 212 and display module 216. The system may present predefined personas such as supervising clinician, front line therapist, care coordinator, and compliance auditor, and it may derive additional traits from organization, location, case type, and device capability. Persona selection 502 writes a signed session profile to database engine 204 that specifies visible sections, editing privileges, evidence detail level, notification preferences, and latency budgets. Communication module 202 distributes the session profile to AI module 210 and ABA module 220 so retrieval depth, decoding constraints, and template fields align with the selected persona.

Access control 504 enforces the session profile by resolving identity, applying role-based permissions, and mediating requests for de-identified versus re-identified content. User module 212 authenticates the user through communication module 202, issues a time-bounded token, and attaches a policy that enumerates permitted operations for each object class stored in database engine 204. Access control 504 filters read and write requests emitted by display module 216 and by external tools, redacts fields at query time when policy requires it, and records an immutable audit event for every granted or denied operation. AI module 210 consults the same policy to block generation of tokens that could reveal personally identifiable information and to restrict retrieval neighborhoods that might surface sensitive context.

Transparency panel 506 presents real-time provenance and rationale to the active persona and collects confirmations when policy requires them. The panel queries database engine 204 for evidence identifiers tied to populated fields and renders the supporting passages, embedding distances, graph paths, and bias metrics returned by AI module 210. The panel also shows which constraints from ABA module 220 and which configuration from governance configuration 512 influenced a recommendation or redaction. When the user expands a given element the panel resolves a direct pointer to the underlying artifact so the user can verify the chain from source record to displayed text without leaving the current view.

Feedback capture 508 converts user actions into structured signals that shape future behavior. The system records accepts, edits, rejections, requests for alternative goals, and provenance acknowledgments as tuples containing the original content, the edited content, the active persona, the evidence set, and the policy version. Feedback capture 508 streams these tuples to AI module 210 for adapter fine tuning and retrieval weight updates, and to ABA module 220 for rule tuning and template binder adjustments. The figure shows a return arrow from feedback capture 508 to persona selection 502 to indicate that accumulated edits may suggest a refined persona preset, such as a preference for more detailed measurement criteria or a narrower evidence scope.

Dashboard 510 aggregates operational and quality metrics for the session and across cohorts so administrators and clinicians can monitor performance and coverage. The dashboard reads counters and traces from database engine 204 and communication module 202 to display ingestion status, model versions, template usage, approval rates, edit magnitudes, bias metrics by attribute, and export outcomes. The dashboard 510 also renders alerts produced by governance configuration 512 when thresholds are approached and provides drill-downs that link to transparency panel 506 for case level inspection. Users may schedule periodic reports from the dashboard that communication module 202 delivers to designated recipients with checksums and signatures.

Governance configuration 512 manages policies that bind the prior stages. Administrators use this stage to define persona templates, access rules, disclosure levels for transparency panel 506, feedback sampling rates, acceptance thresholds for recommendation scoring, and bias mitigation actions. Governance configuration 512 writes policy bundles to database engine 204 with semantic version numbers and distributes hashes to AI module 210, ABA module 220, user module 212, and display module 216. The figure shows a side arrow from transparency panel 506 to governance configuration 512 to reflect that policy can tighten or relax disclosure based on observed usage and regulatory updates, and it shows that new governance settings take effect in subsequent sessions through persona selection 502 and access control 504.

Usability testing and iteration 514 closes the loop by validating that configured policies and user experiences achieve desired outcomes without undue friction. This stage orchestrates A/B experiments, task time measurements, error rate tracking, and satisfaction surveys embedded in display module 216. Usability testing and iteration 514 analyzes the captured telemetry and survey responses stored in database engine 204, compares them against acceptance criteria set in governance configuration 512, and proposes changes to template layouts, transparency defaults, evidence density, and interaction flows. The figure indicates a feedback arrow that returns to earlier stages to implement those changes so that subsequent sessions begin with refined personas, updated access policies, and improved transparency and feedback mechanisms.

Together these stages provide a governed interaction pipeline that tailors outputs to a defined persona, enforces privacy and privilege, exposes provenance and rationale, captures structured feedback for learning, visualizes quality and performance, applies configurable policies, and iterates the experience based on measured usability. Each stage reads and writes versioned artifacts in database engine 204, uses communication module 202 for secure transport, and coordinates with AI module 210 and ABA module 220 so technical processing aligns with policy and user intent throughout the lifecycle.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer executable instructions of a computer program product running on a computer or computers, and those skilled in the art would recognize that this disclosure can be implemented in combination with other program modules. Generally, program modules include routines, programs,

components, data structures, etc. that perform particular tasks and/or implement particular abstract data types. Those skilled in the art would appreciate that the computer-implemented methods disclosed herein can be practiced with other computer system configurations, including single processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions according to the processor architecture until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A computer-implemented method for generating an applied behavior analysis report, the method comprising:
aggregating and normalizing, by one or more processors, applied behavior analysis data from structured and unstructured sources comprising at least electronic health records, case studies, clinical assessments, and user inputs;
anonymizing, by the one or more processors, personally identifiable information contained in the applied behavior analysis data;
processing, by the one or more processors executing an artificial intelligence model, the anonymized applied behavior analysis data using natural language processing to perform tokenization, lemmatization or stemming, part-of-speech tagging, and named entity recognition;
generating, by the one or more processors, contextual embeddings using a transformer based architecture to derive semantic relationships among observed behaviors, assessment findings, interventions, goals, and outcomes in an applied behavior analysis domain;
detecting, by the one or more processors, bias in model outputs and adjusting a recommendation score responsive to the detected bias; selecting, by the one or more processors, a report template and auto-populating fields of the report template with individualized goals, interventions, and guidance based on the contextual embeddings and the adjusted recommendation score; and
outputting, by the one or more processors, a personalized applied behavior analysis report comprising an individualized intervention and behavior intervention guide.

2. The method of claim 1, wherein aggregating and normalizing the applied behavior analysis data comprises ingesting longitudinal records associated with a client across multiple sessions and normalizing the records into a unified case context used for the report generation.

3. The method of claim 1, wherein anonymizing the personally identifiable information comprises detecting and removing or obfuscating identifiers prior to processing the applied behavior analysis data using the artificial intelligence model.

4. The method of claim 1, wherein the applied behavior analysis data further comprises behavioral observations captured during an immersive session presented through augmented reality or virtual reality interface.

5. The method of claim 4, wherein the immersive session captures sensor data comprising at least one of eye gaze, head or body movement, gesture interaction, reaction time, or vocalizations, and wherein the sensor data is converted into feature representations used to generate the contextual embeddings.

6. The method of claim 1, wherein the applied behavior analysis data further comprises behavioral observations captured through passive environmental sensing without requiring a subject to wear an immersive device.

7. The method of claim 6, wherein the passive environmental sensing comprises acquiring data from at least one of ambient cameras, microphones, depth sensors, or wearable devices, and transforming the data into de-identified behavioral features prior to processing by the artificial intelligence model.

8. A decision-path selection system comprising:
one or more processors: and
non-transitory memory storing instructions that, when executed by the one or more processors, cause the system to:
(a) receive a candidate set comprising potential goals, interventions, actions, or recommendations represented by feature vectors;
b) receive a plurality of constraints comprising at least one of clinical, administrative, policy-based, sequencing, or coverage constraints;
(c) construct an optimization model from the candidate set and the plurality of constraints, the optimization model comprising variables and an objective function;
(d) submit the optimization model to a solver selected from a classical solver, a quantum solver, a quantum-simulated solver, or a quantum-inspired solver;
(e) receive one or more candidate solutions from the solver;
(f) verify the one or more candidate solutions against the plurality of constraints; and
(g) select or rank one or more candidates based on verified solutions,
wherein the selected or ranked candidates are output for downstream use by another computing component or software module.

9. The system of claim 8, wherein constructing the optimization model comprises encoding the objective function and the plurality of constraints as a quadratic unconstrained binary optimization formulation or a bounded integer optimization formulation.

10. The system of claim 8, wherein submitting the optimization model comprises selecting the solver based on at least one of execution time, computational cost, or solution quality constraints.

11. The system of claim 8, wherein verifying the one or more candidate solutions comprises re-evaluating each candidate solution against the plurality of constraints prior to permitting selection.

12. The system of claim 8, further comprising persisting one or more audit artifacts associated with the optimization model, solver configuration, or selected candidates, wherein the audit artifacts enable offline reproduction of the selection.

13. The system of claim 8, wherein verification comprises rejecting candidate solutions that violate mandatory constraints and selectively relaxing secondary objectives under a predefined policy that prohibits constraint breaches.

14. A behavioral data capture system comprising: one or more processors; a virtual-reality or augmented-reality interface configured to present interactive content to a user; and
non-transitory memory storing instructions that, when executed by the one or more processors, cause the system to:
(a) capture behavioral signals generated by the user during interaction with the interactive content using one or more sensors integrated with the interface;
(b) temporally align the behavioral signals with session events occurring within the interactive content; and
(c) generate feature representations from the behavioral signals for downstream behavioral analysis.

15. The system of claim 14, wherein the behavioral signals comprise at least one of gaze direction, head motion, body posture, gesture, vocalization, response latency, or physiological measurements.

16. The system of claim 14, wherein the feature representations are generated in real time during the interactive session.

17. A passive behavioral sensing system comprising: one or more environmental sensors positioned within a physical environment; and non-transitory memory storing instructions that, when executed by one or more processors, cause the system to:
(a) capture behavioral observations of a subject without requiring the subject to wear an immersive interface;
(b) preprocess the behavioral observations to remove or obfuscate personally identifiable information; and
(c) generate de-identified behavioral feature representations aligned with contextual or session metadata.

18. The system of claim 17, wherein the environmental sensors comprise at least one of ambient cameras, depth sensors, microphones, or wearable devices.

19. The system of claim 17, wherein the behavioral observations comprise at least one of locomotion patterns, interaction behaviors, facial expressions, vocalizations, or physiological responses.

20. The system of claim 17, wherein immersive behavioral capture and passive environmental sensing are selectively employed independently or in combination within the same behavioral analysis platform.
